(19) United States
(12) Patent Application Publication
(10) Pub. No.: US 2026/0076825 A1
(43) Pub. Date: Mar. 19, 2026

(54) System and Method for Applying Eye Drops

(71) Applicant: Alexa Shlien, Ville St Laurent (CA)

(72) Inventor: Alexa Shlien, Ville St Laurent (CA)

(21) Appl. No.: 18/885,701

(22) Filed: Sep. 15, 2024

(51) Int. Cl. A61F 9/00

(52) U.S. Cl. CPC A61F 9/0026

(57) ABSTRACT

Embodiments of the present invention may comprise a system and method for applying eye drops. An eye mask may have a first eye cover and a second eye cover that are configured over a user's eyes. Each eye cover may have an eye drop bottle receiver that allows an eye drop bottle to be removably connected to the eye covers. The eye drop bottle may contain an eye drop fluid. When the eye drop bottle is squeezed, some of the eye drop fluid may be forced from the eye drop bottle through an eye drop bottle receiver and into a user's eye, thereby efficiently applying eye drops to the user's eye without having to aim the eye drop bottle.

### BACKGROUND OF THE INVENTION

Embodiments of the present invention may comprise a system and/or method for applying eye drops. Eye drops a generally applied to one's eyes via an eye dropper or eye drop bottle. To apply eye drops using these devices, a user must aim the eye dropper or eye drop bottle so that the eye drops are applied directly to the user's eyes. This can be difficult and may result in accidentally applying eye drops to portions of a user's face other than their eyes, which wastes the eye drops and does not alleviate the user's eye condition. Furthermore, applying eye drops can be a stressful experience since people naturally do not want to introduce foreign objects, including eye drops, into their eyes. This makes it even more difficult to apply eye drops directly to one's eye using a standard eye dropper or eye drop bottle.

### SUMMARY OF THE INVENTION

Embodiments of the present invention comprise a system for applying eye drops. An eye mask may have a first eye cover and a second eye cover. Each eye cover may have an eye drop bottle receiver. Each eye drop bottle receiver may comprise an opening that allows objects such as eye drop fluid to pass through its respective eye cover into a user's eye. The first eye cover and second eye cover may be connected by a connector band whereby the connector band is configured between the first eye cover and the second eye cover. A headband may also connect to the first eye cover and the second eye cover. The headband may be significantly longer than the connector band since the headband is meant to secure the eye mask to a user's head rather than serve as a structural connection between the first eye cover and second eye cover.

A first eye cover flange may be connected to the first eye cover. A second eye cover flange may be connected to the second eye cover. The headband may be connected to the first eye cover via the first eye cover flange. The headband may be connected to the second eye cover via the second eye cover flange. Each the first eye cover flange and the second eye cover flange may have at least one opening through which the headband is configured to secure the headband to the first eye cover flange and to the second eye cover flange. The first eye cover flange may extend from the first eye cover in a direction opposite the connector band. The second eye cover flange may extend from the second eye cover in a direction opposite the connector band.

An eye drop bottle of the system may contain an eye drop fluid. The eye drop fluid may be a medicinal substance intended to be applied to a user's eyes. The eye drop fluid may be a saline solution. The eye drop fluid may alternatively have other active ingredients such as but not limited to polyethylene glycol, polyvinyl alcohol, propylene glycol, carboxymethylcellulose, povidone, glycerin, and mineral oil.

The eye mask may be donned by a user. The first eye cover and second eye cover may be configured to cover the user's eyes. The first eye cover and second eye cover may cover a larger area of the user's face than just the user's eyes. The eye drop bottle receiver of each eye cover may be configured directly over a user's eye so that liquid passing through the eye drop bottle receiver is applied to the user's eye. The headband may be configured around the user's head to secure the eye mask to the user's head. When the eye mask is donned by a user, the connector band may be configured over the bridge of the user's nose.

The eye drop bottle may be removably connected to the first eye cover or the second eye cover via the respective eye drop bottle receivers. The eye drop bottle may have a base end and an open end. The open end may have an opening so that eye drop fluid may escape the eye drop bottle to be applied to a user's eyes. The open end of the eye drop bottle may have external threads that mate with corresponding internal threads of the eye drop bottle receivers to removably connect the eye drop bottle to the eye drop bottle receivers. Alternatively, the open end of the eye drop bottle may have a male taper that mates with a corresponding female taper of the eye drop bottle receivers to removably connect the eye drop bottle to the eye drop bottle receivers. Alternatively, the open end of the eye drop bottle may have male Luer threads that mate with corresponding female Luer threads of the eye drop bottle receivers to removably connect the eye drop bottle to the eye drop bottle receivers. Luer threads are defined herein as the threaded portions of a Luer lock. In addition to the male Luer threads, the open end of the eye drop bottle may also have a male taper that mates with a corresponding female taper of the eye drop bottle receivers to form Luer locks with the eye drop bottle receivers. In such embodiments, the Luer threads of the eye drop bottle and eye drop bottle receivers may be designed such that 2 or less rotations of the eye drop bottle within the eye drop bottle receivers are sufficient to removably connect the eye drop bottle to the eye drop bottle receivers.

The eye drop bottle receivers of the first eye cover and the second eye cover may each have an eye drop bottle receiver extrusion that protrudes from its respective eye cover away from the user's eyes when the eye mask is donned by the user. Each eye drop bottle receiver extrusion may have an opening so that eye drop fluid may pass through the eye drop bottle receiver extrusion. In embodiments wherein the eye drop bottle receivers comprise internal threads, the internal threads may be configured on inner surfaces of the eye drop bottle receiver extrusions. In embodiments wherein the eye drop bottle receivers comprise female tapers, the inner surfaces of the eye drop bottle receivers may be tapered. Alternatively, the entirety of each eye drop bottle receiver extrusion may be tapered. In embodiments wherein the eye drop bottle receivers comprise female Lue threads, the female Luer threads may be configured on the inner surfaces of the eye drop bottle receiver extrusions.

The eye drop bottle may have a side surface configured between the base end and the open end. The side surface may be an endless rectangle such that the eye drop bottle is cylindrical. The side surface may deform when a compression force is applied to points of the side surface from outside the eye drop bottle. This may be referred to as the eye drop bottle being “squeezed”. When the eye drop bottle is squeezed, the deformation of the side surface may force eye drop fluid out of the eye drop bottle via the open end. When the eye drop bottle is removably connected to the first eye cover or second eye cover via an eye drop bottle receiver and when the eye mask is donned by a user, the eye drop fluid may pass through the eye drop bottle receiver and into a user's eye, thereby applying the eye drop fluid to the user's eye using the system for applying eye drops.

The configuration of the eye drop bottle receivers directly over the user's eyes, along with the removable connection between the eye drop bottle and the eye drop bottle receivers makes it easier for the user to apply eye drops since the user does not have to aim the eye drop bottle at their eyes. Furthermore, any stress, anxiety, or fear felt by the user when applying eye drops will not affect the ability of the user to aim the eye drop bottle since the eye drop bottle is secured to the face mask, which is secured to the user's face. Therefore, if the user's hand is shaking due to emotion when squeezing the eye drop bottle, the shaking of the user's hand will have no effect on the location of the eye drop bottle relative to the user's eyes.

The base end of the eye drop bottle may be completely closed so that eye drop fluid does not escape the eye drop bottle via the base end. The eye drop bottle may have a removable cover that may be removably connected to the open end of the eye drop bottle. The removable cover may be removed prior to removably connecting the eye drop bottle to an eye drop bottle receiver. The removable cover may be removably connected to the open end of the eye drop bottle via internal threads that mate with corresponding external threads of the eye drop bottle. The removable cover may alternatively be removably connected to the open end of the eye drop bottle via female Luer threads that mate with corresponding male Luer threads of the eye drop bottle. The removable cover may alternatively be removably connected to the open end of the eye drop bottle via an adhesive.

Embodiments of the present invention may also comprise a method for applying eye drops. An eye mask may be provided. The eye mask may be the same eye mask or a similar eye mask to that described in the system for applying eye drops herein. An eye drop bottle containing eye drop fluid may be provided. The eye drop bottle may be the same eye drop bottle or a similar eye drop bottle to that described in the system for applying eye drops herein. The eye drop fluid may be the same eye drop fluid or a similar eye drop fluid to that described in the system for applying eye drops herein.

The eye mask may be donned by a user by configuring the headband of the eye mask around the user's head whereby the first eye cover and the second eye cover are configured over the user's eyes. The first eye cover and the second eye cover may cover a greater portion of the user's face than just the user's eyes. The eye drop bottle may be removably connected to the first eye cover or to the second eye cover via the respective eye drop bottle receiver. The eye drop bottle may be squeezed as described herein to release eye drop fluid from the eye drop bottle through the eye drop bottle receiver into one of the user's eyes.

In embodiments wherein the eye drop bottle has a removable cover, the removable cover may be removed prior to removably connecting the eye drop bottle to the first eye cover of the second eye cover.

### BRIEF DESCRIPTION OF DRAWINGS

FIG. 1 shows a top front perspective view of an eye mask of a system for applying eye drops.

FIG. 2 shows a top front perspective view of a system for applying eye drops.

FIG. 3 shows a top view of a system for applying eye drops.

### DETAILED DESCRIPTION OF THE INVENTION

The description provided herein describes example embodiments of the present invention and is not intended to limit the invention to any particular embodiment, component, feature, size, shape, design, material, or any other property. Furthermore, the drawings provided herein show example embodiments of the present invention and are not intended to limit the invention to any particular embodiment, component, feature, size, shape, design, material, or any other property.

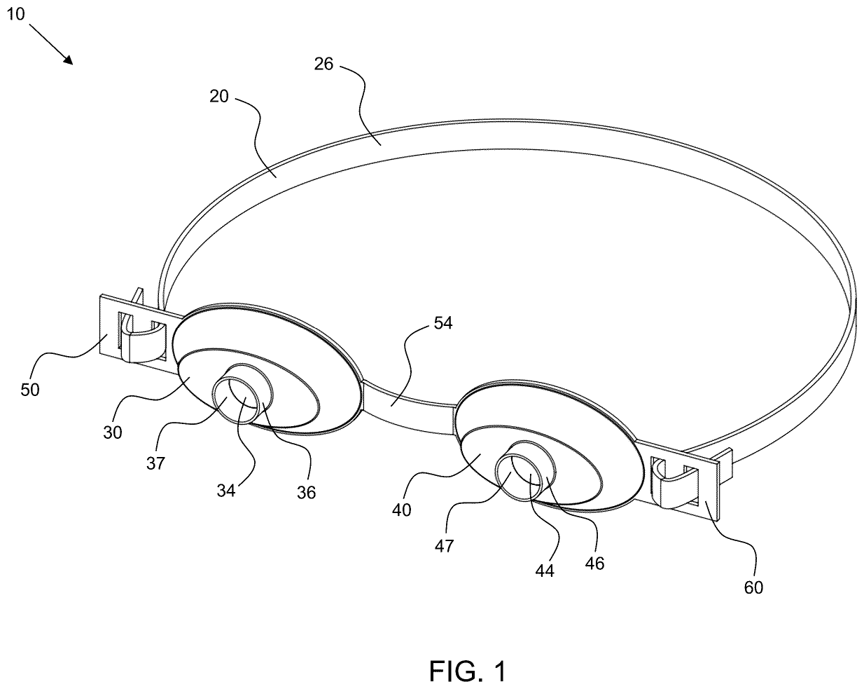

As shown in FIG. 1, an eye mask 20 of a system for applying eye drops 10 has a first eye cover 30 and a second eye cover 40. A connector band 54 connects the first eye cover 30 and second eye cover 40. A first eye cover flange 50 extends from the first eye cover 30 in a direction opposite the connector band 54. A second eye cover flange 60 extends from the second eye cover 40 in a direction opposite the connector band 54.

The first eye cover flange 50 and the second eye cover flange 60 each have two slots through which a headband 26 is configured to secure the headband 26 to the first eye cover flange 50 and the second eye cover flange 60. When the eye mask 20 is donned by a user (not shown), the headband 26 is configured around the user's head such that the first eye cover 30 and the second eye cover 40 cover the user's eyes. The headband 26 may be made of a material with a Young's Modulus of 0.1 GPa or less such as but not limited to rubber or silicone. This allows the headband 26 to stretch to accommodate the shape and size of a user's head and then retract to secure the eye mask 20 to the user's head. The connector band 54, first eye cover flange 50, and second eye cover flange 60 may be made of the same material as the headband 26. Alternatively, the connector band 54, first eye cover flange 30, and second eye cover flange 60 may be made of a material with a greater Young's Modulus than that of the headband 26. This allows the eye mask 20 to better retain its shape since the first eye cover flange 50, second eye cover flange 60, and connector band 54 may be structural components of the eye mask 20.

The first eye cover 30 has an eye drop bottle receiver 34 that extends through the first eye cover 30 such that an eye drop fluid may be applied to a user's eye through the first eye cover 30 via the eye drop bottle receiver 34. An eye drop bottle receiver extrusion 36 extends from the first eye cover 30 in a direction opposite the headband 26 when the headband 26 is secured around a user's head. The eye drop bottle receiver extrusion 36 has an inner surface 37 on which internal threads or other connection features may be configured. The second eye cover 40 also has an eye drop bottle receiver 44 that extends through the second eye cover 40 such that an eye drop fluid may be applied to a user's eye through the second eye cover 40 via the eye drop bottle receiver 44. An eye drop bottle receiver extrusion 46 extends from the second eye cover 40 in a direction opposite the headband 26 when the headband 26 is secured around a user's head. The eye drop bottle receiver extrusion 46 has an inner surface 47 on which internal threads or other connection features may be configured.

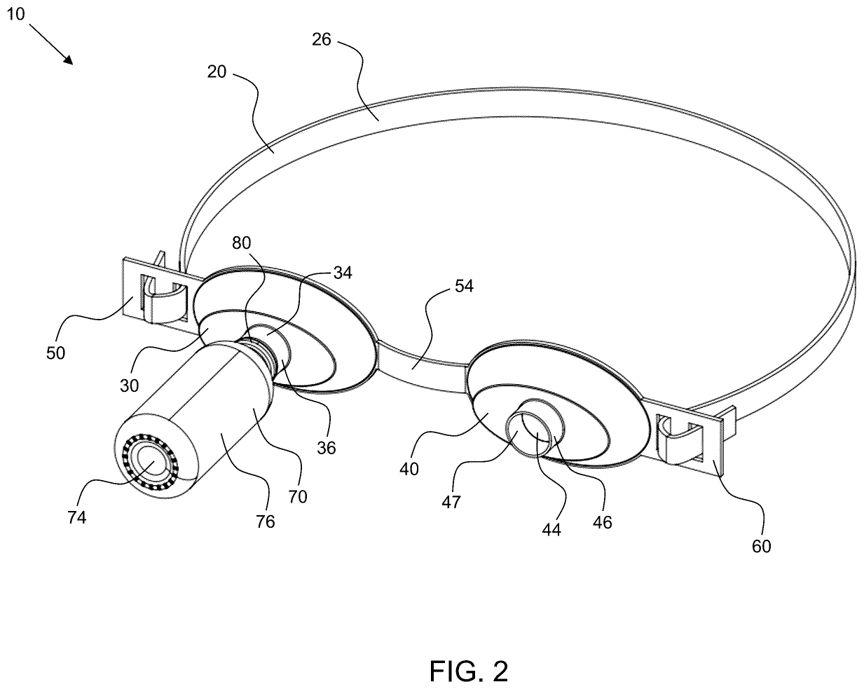

As shown in FIG. 2, an eye drop bottle 70 is removably connected to the first eye cover 30 via the eye drop bottle receiver 34. More specifically, the eye drop bottle 70 is connected to the first eye cover 30 via the eye drop bottle receiver extrusion 36 of the eye drop bottle receiver 34. The eye drop bottle 70 has a base end 74 and an open end 80 configured opposite the base end 74. The open end 80 of the eye drop bottle 70 is removably connected to the eye drop bottle receiver extrusion 36 such that an eye drop fluid (not shown) contained within the eye drop bottle 70 may pass from the eye drop bottle 70 through the open end 80 and pass through the first eye cover 30 through the eye drop bottle receiver 34.

The eye drop bottle 70 has a side surface 76. The side surface is an endless rectangle such that the eye drop bottle 70 is cylindrical. As shown in FIG. 2, the eye drop bottle 70 may not be a perfect cylinder. However, the eye drop bottle 70 may be considered cylindrical since the side surface 76 is an endless rectangle and since the base end 74 is configured perpendicular the side surface 76.

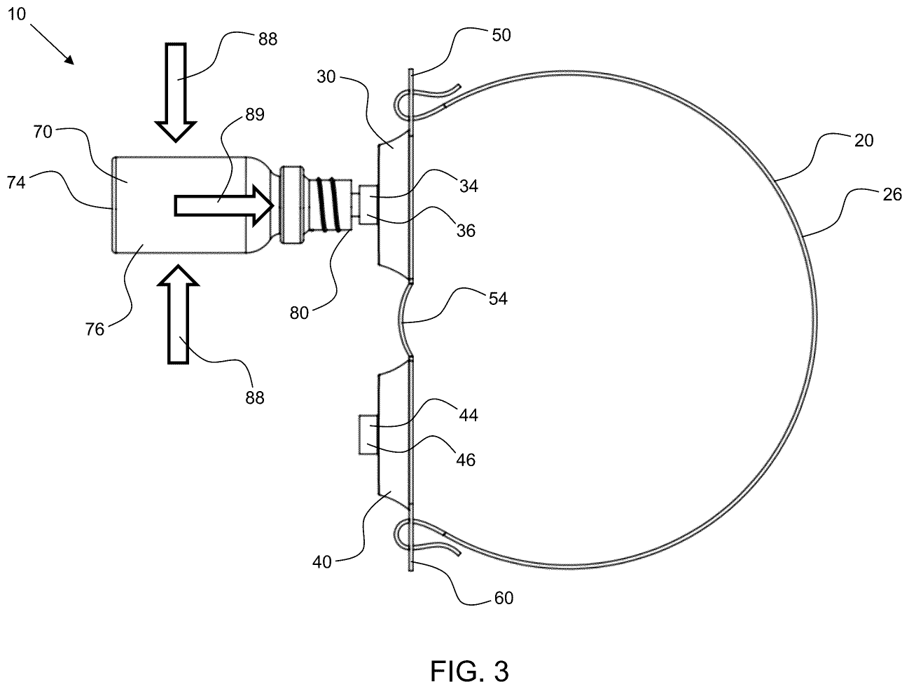

As shown in FIG. 3, the eye drop bottle 70 is removably connected to the first eye cover 30 via the open end 80 of the eye drop bottle 70 and the eye drop bottle receiver 34 of the first eye cover 30.

Eye drop fluid is released from the eye drop bottle 70 when the eye drop bottle 70 is squeezed. The arrows 88 shown in FIG. 3 show the direction in which the side surface 76 of the eye drop bottle 70 is squeezed to release eye drop fluid from the eye drop bottle 70. The arrow 89 shown in FIG. 3 shows the direction in which eye drop fluid travels after the side surface 76 of the eye drop bottle 70 is squeezed to release the eye drop fluid via the open end 80. The open end 80 of the eye drop bottle 70 may have an opening small enough that eye drop fluid does not escape the eye drop bottle 70 until the eye drop bottle 70 is squeezed to force eye drop fluid through the opening. In this manner, when using the system for applying eye drops 10 the user may configure their head such that the base end 74 of the eye drop bottle 70 is pointed upwards such that eye drop fluid flows to the open end 80 of the eye drop bottle 70 due to the force of gravity. The eye drop fluid then flows through the eye drop bottle receiver 34 into the user's eye due at least partially to the force of gravity when the eye drop bottle 70 is squeezed.

The eye drop bottle 70 may be made of a material with a Young's Modulus of 0.5 GPa or less. This allows the eye drop bottle 70 to be easily squeezed to release the eye drop fluid. The eye drop bottle 70 may also be made of a material with a Young's Modulus of 0.1 GPa or greater. This allows the eye drop bottle 70 to better retain its shape when not squeezed. The eye drop bottle 70 may be made of a biocompatible material such as polyethylene or the like. “Biocompatible material” is defined herein as a material generally accepted to be safe for medical devices such as eye droppers.

1. A system for applying eye drops comprising:
an eye mask comprising:
a first eye cover comprising an eye drop bottle receiver;
a second eye cover comprising an eye drop bottle receiver;
a connector band configured between the first eye cover and the second eye cover;
a headband connected to the first eye cover and the second eye cover; and
an eye drop bottle containing an eye drop fluid,
wherein the eye mask is donned by a user by configuring the first eye cover and the second eye cover over the user's eyes and configuring the headband around the user's head,
wherein the eye drop bottle is removably connected to the first eye cover via the eye drop bottle receiver,
wherein the eye drop bottle is squeezed to release eye drop fluid from the eye drop bottle through the eye drop bottle receiver into a user's eye.

2. The system of claim 1, wherein the eye drop bottle comprises external threads,
wherein the eye drop bottle receiver comprises internal threads,
wherein the eye drop bottle is removably connected to the first eye cover by mating the external threads of the eye drop bottle to the internal threads of the eye drop bottle receiver.

3. The system of claim 1, wherein the eye drop bottle comprises male Luer threads,
wherein the eye drop bottle receiver comprises female Luer threads,
wherein the eye drop bottle is removably connected to the first eye cover by mating the male Luer threads of the eye drop bottle to the female Luer threads of the eye drop bottle receiver.

4. The system of claim 1, wherein the eye drop bottle comprises a male taper,
wherein the eye drop bottle receiver comprises a female taper,
wherein the eye drop bottle is removably connected to the first eye cover by mating the male taper of the eye drop bottle to the female taper of the eye drop bottle receiver.

5. The system of claim 1, wherein the eye drop bottle comprises:
a base end;
and open end; and
a side surface,
wherein the side surface is squeezed to release eye drop fluid from the eye drop bottle through the eye drop bottle receiver into a user's eye.

6. The system of claim 5, wherein the side surface is an endless rectangle,
wherein the eye drop bottle is cylindrical.

7. The system of claim 1, further comprising:
a first eye cover flange connected to the first eye cover; and
a second eye cover flange connected to the second eye cover,
wherein the headband is connected to the first eye cover via the first eye cover flange,
wherein the headband is connected to the second eye cover via the second eye cover flange.

8. The system of claim 1, wherein each eye drop bottle receiver comprises an eye drop bottle receiver extrusion that protrudes from its respective eye cover away from the user's eyes.

9. The system of claim 1, wherein the eye drop bottle comprises a removable cover,
wherein the removable cover is removed prior to removably connecting the eye drop bottle to the first eye cover.

10. The system of claim 1, wherein the eye drop fluid is a saline solution.

11. A method for applying eye drops comprising:
providing an eye mask comprising:
a first eye cover comprising an eye drop bottle receiver;
a second eye cover comprising an eye drop bottle receiver;
a connector band configured between the first eye cover and the second eye cover;
a headband connected to the first eye cover and the second eye cover;
providing an eye drop bottle containing eye drop fluid;
donning the eye mask by configured the headband around a user's head whereby the first eye cover and the second eye cover are configured over the user's eyes;
removably connecting the eye drop bottle to the first eye cover via the eye drop bottle receiver; and
squeezing the eye drop bottle wherein eye drop fluid is released from the eye drop bottle through the eye drop bottle receiver into a user's eye.

12. The method of claim 11, wherein the eye drop bottle comprises external threads,
wherein the eye drop bottle receiver comprises internal threads,
wherein the eye drop bottle is removably connected to the first eye cover by mating the external threads of the eye drop bottle to the internal threads of the eye drop bottle receiver.

13. The method of claim 12, wherein the eye drop bottle comprises male Luer threads,
wherein the eye drop bottle receiver comprises female Luer threads,
wherein the eye drop bottle is removably connected to the first eye cover by mating the male Luer threads of the eye drop bottle to the female Luer threads of the eye drop bottle receiver.

14. The method of claim 11, wherein the eye drop bottle comprises a male taper,
wherein the eye drop bottle receiver comprises a female taper,
wherein the eye drop bottle is removably connected to the first eye cover by mating the male taper of the eye drop bottle to the female taper of the eye drop bottle receiver.

15. The method of claim 12, wherein the eye drop bottle comprises:
a base end;
and open end; and
a side surface,
wherein the side surface is squeezed to release eye drop fluid from the eye drop bottle through the eye drop bottle receiver into the user's eye.

16. The method of claim 15, wherein the side surface is an endless rectangle,
wherein the eye drop bottle is cylindrical.

17. The method of claim 11, wherein the eye mask further comprises:
a first eye cover flange connected to the first eye cover; and
a second eye cover flange connected to the second eye cover,
wherein the headband is connected to the first eye cover via the first eye cover flange,
wherein the headband is connected to the second eye cover via the second eye cover flange.

18. The method of claim 11, wherein each eye drop bottle receiver comprises an eye drop bottle receiver extrusion that protrudes from its respective eye cover away from the user's eyes.

19. The method of claim 11, wherein the eye drop bottle comprises a removable cover,
wherein the method further comprises removing the removable cover prior to removably connecting the eye drop bottle to the first eye cover.

20. The method of claim 11, wherein the eye drop fluid is a saline solution.
